(19) United States
(12) Patent Application Publication
(10) Pub. No.: US 2009/0035806 A1
(43) Pub. Date: Feb. 5, 2009

(54) Method for Identification of Age of an Ancient Ware by Using Microbes

(76) Inventor: Jiajun Tian, Hangzhou (CN)

Correspondence Address: GLOBAL IP SERVICES, 7285 W. Eagle Court, Winton, CA, 95388, US

(21) Appl. No.: 12/180,757

(22) Filed: Jul. 28, 2008

(30) Foreign Application Priority Data
Aug. 1, 2007 (CN) 200710070376.X

(51) Int. Cl. C12Q 1/02

(52) U.S. Cl. 435/29

(57) ABSTRACT

This invention is related to a method for identification of age of the ancient wares, particularly a method for identification of age of the ancient wares by using the microbe. The method of this invention includes the following steps: establish database corresponding to the microbe parameters in wares to be measured; take sample from the wares to be measured; measure the microbe parameters value; compare the obtained parameters value with the standard database to obtain the ware production year. This invention has features of correct and reliable identification of age of the ancient wares, and wide application range.

### CROSS REFERENCE TO THE RELATED PATENT APPLICATION

This application claims the priority of the Chinese patent application No. 200710070376.X with a filing date of Aug. 1, 2007, which application is incorporated herein by reference.

### FIELD OF THE INVENTION

This invention is related to a method for identification of age of the ancient wares, particularly a method for identification of age of the ancient wares by using the microbe.

### BACKGROUND OF THE INVENTION

The jade article, handed down from remote ancient times, means jade article left over from various kinds of cultural relics since Neolithic Era, going around the world after unearthing at various times and from various places. The feature of the most of the jade articles is left from the unearthing place and environment bringing about many difficulties for the identification of certain jade articles, handed down from remote ancient times. Therefore, it is very important to find how to proceed with identification and the reasonable identification steps. The first is to judge if they are real remote ancient jade articles or false remote ancient jade articles. Without judging, there is no talk about others.

Currently, identification of the genuine or forged jade article, handed down from remote ancient times is resting on the experience identification phase. It is the experience summing-up like a kind of secret recipe: to judge the truth of certain jade article, handed down from remote ancient times relying on individual rich experience, knowledge and vision of a few experts. It is often difficult to check the summing-up obtained from comparison with general knowledge. Because the experts of identification explain the real or false ancient jade according to their own experience and general knowledge, sometimes there is very big difference in such identification summing-up. If one kind of phenomenon has some different explanations, and cannot be verified, such summing-up is the only saying of one expert, and is very difficult to be regarded as the scientific one. Such summing-up obtained by the position of the scholar, experience and seniority used as a basis of judgment is titled with the scientific name, but it is contrary to the original scientific sense. Handling complicated scientific problem with experience, general knowledge and traditional method of intuitive thinking is not adequate to the current time. It not only restricted the research depth and breadth, but also sometimes may lead to our research and identification of real or false jade articles, handed down from remote ancient times to a wrong way.

The Chinese patent publication No. 1232857 provided a method for identification of sequence of crossing and overlapping of the writing trace and the printed text. Its characteristic includes the following steps: 1. Take picture of the material to be identified under the microscope forming the microscopic photographic image; 2. Establish the test sample according to different modes formed according to crossing and overlapping of the writing trace and the printed text, and take a picture of the test sample under the microscope so as to form the microscopic photographic image; 3. Compare characteristic of the microscopic photographic image obtained from step 1 with characteristic of the microscopic photographic image obtained from step 2, and find characteristic of the microscopic photographic image, obtained from the test material, equal to the microscopic photographic image formed from the test sample, thus to obtain sequence of crossing and overlapping of the writing trace and the printed text. In process of establishing the microscopic photographic image of the test sample, according to different inks for writing (including carbon ink, blue-black ink, ball pen oil and carbon paper autotype form), sequence of crossing and overlapping of the writing trace and the printed text (first write and then stamp seal, first stamp seal and then write), time duration (immediately write after stamping seal, write after drying of the printed text), separately establish the test sample to form comparative microscopic photographic image. In process of comparison, carry out characteristic comparison of microscopic photographic image of the test material obtained from step 1 with microscopic photographic image of the test material obtained from step 2, and find characteristic of the test sample with equal microscopic photographic image formed from the test material, i.e. the microscopic photographic image of “blue-black ink, first stamp seal and then write”, thus to obtain sequence of crossing and overlapping of the writing trace and the printed text, i.e.: first stamp seal and then write. Good effects were reached in using this method to identify sequence of crossing and overlapping of the printed text, but cannot be used to identify the real or false jade article, handed down from remote ancient times.

The Chinese patent application No. 200610090931.0 published a computer-aided method for judging generation, namely: the image pick-up equipment includes digital camera or microscopic photography equipment; the image storage and comparison equipment is computer and PHOTOSHOP software used for image storage and comparison. The real and false identification method and step of the jade article, handed down from remote ancient times is as follows: A. Establish photographic electronic database of non-primordial characteristic of the unearthed ancient jade: a. Select the whole unearthed ancient jade with structural characteristic and select 0.5˜2 cm² of the object block with surface elementary characteristic, and use digital camera for taking picture; the digital camera should have not less than 5 million pixels. The digital camera for microscopic photography feeds the digitalized image with non-primordial characteristic into the computer and storages in the computer. b. Establish database of non-primordial characteristic of the unearthed ancient jade by using the surface layer non-primordial characteristic image of the unearthed ancient jade, classify according to types of the surface layer non-primordial characteristic image of the unearthed ancient jade, and connect every characteristic element with the corresponding image, so as to be able to open and operate it in PHOTOSHOP software environment. B. Select the surface layer non-primordial element characteristic image of the jade to be identified through amplifying glass, and shoot it into microscopic digital electronic image. These characteristics have important sense for identification of genuineness and falsehood. The mentioned amplifying glass should be of 20 times. Feed the microscopic digital electronic image of the jade to be identified into computer, so as to be able to open and operate it in PHOTOSHOP software environment. The mentioned selected surface layer non-primordial element characteristic of the jade to be identified includes identification of all structural characteristics and the surface layer non-primordial element characteristic of the jade to be identified. C. Open the microscopic image of the jade to be identified in computer, and compare it with the standard sample of the ancient jade in database; if the image element characteristics are the same, the jade to be identified is real, if are not the same, the jade to be identified is false.

### SUMMARY OF THE INVENTION

This invention solved the problems existing in incorrect identification of years, low reliability and other problems, providing a method of identification of years of the ancient wares by using the microbe.

The above-mentioned technical purpose of this invention was reached through the following technical schemes: method of using the microbe for identification of the ancient wares; it includes the following steps:

A. Establish database corresponding with the microbe parameters in the wares to be measured;

B. Take and check the microbe sample from the wares to be measured;

C. Measure the microbe parameters value;

D. Compare the obtained parameters value with the standard parameters value in database to obtain the production year of the ware.

Trace of the microbe left on the ancient ware is the basis reflecting years of the ancient ware. The color on the ancient jade, as aim of this invention, is result of the product in long-term growth, metabolism and reproduction of the microbe. Therefore, it is possible to judge years of the ancient jade through method of observing color by human eyes. However, it needs experience of the observer, and the reliability is not high. The inventor discovered that the spacing on the ancient jade enables the microbe to live freely in the spacing. With long time of the microbe living in the ancient jade, its growth, metabolism and reproduction created the color difference. However, the metabolism products of the microbe living in the ancient jade can reflect year of the microbe. Through establishing database of the concrete microbe parameters of the ancient jade of the known year, it is possible to obtain the table of years of the concrete corresponding microbe parameters of the ancient jade. Later on, when fall coming across the ancient jade, it is only necessary to measure the concrete corresponding parameters of the ancient jade with the same microbe of the ancient jade to be measured, and then from the corresponding database it is possible to comparatively find the year corresponding to the concrete parameters of the concrete microbe of the ancient jade. This year is the production year of that ancient jade. At the same time, this invention can also be used for identification of other ancient jades, e.g. bronze wares, engraved stone wares etc. The damage from engraving on their surface might leave trace for the microbe, thus providing possibility for application of the method described in this invention.

As optimization, aspergillus and other microbes can be selected for the mentioned microbe. The year reflected by using physical and/or chemical data of this bacterium is relatively correct.

As optimization, parameters of the mentioned microbe are physical and/or chemical parameters of the microbe debris.

As optimization, the color change parameters of the microbe debris are selected as parameters of the mentioned microbe.

As optimization, the corrosion parameters of the microbe are selected as parameters of the mentioned microbe.

As optimization, the mentioned microbe parameters value is measured by using electronic scanning instrument and so on.

Through the above-mentioned identification method, special measuring instruments and equipment are researched, developed and used for measuring the various mentioned parameters described in this invention.

Therefore, this invention has features of correct and reliable identification of year of the ancient wares.

### DETAIL DESCRIPTION OF THE INVENTION

Through the concrete case, the technical scheme of this invention is further described as follows.

Case 1: This invention uses method of the above-mentioned microbe for identification of the ancient jade with the following steps: search the ancient jade produced in the known year from 2000 B.C. to 1850 A.D., and then use electronic scanning instrument for selecting the residual products of aspergillus and other microbes on the ancient jade, take sample and measure physical and chemical parameters data of the residual products. Prepare table of the above-mentioned measured values and their corresponding years, and connect the obtained point data into line to obtain the picture and database of the residual products of aspergillus and other microbes corresponding to year of the ancient jade. Under the same conditions, measure the corresponding parameters of aspergillus and other microbes on the ancient jade to be identified, compare the obtained corresponding pictures, and then it is possible to know the production year corresponding to the ancient jade, thus the ancient jade identification is completed.

Case 2: This invention adopts the above-mentioned method for identifying year of the engraved jade with the following steps: search the ancient jade produced in the known year from 2000 B.C. to 1850 A.D., and then use electronic scanning instrument for selecting the residual products of aspergillus and other microbes on the ancient jade, take sample and measure physical and chemical parameters data of the metabolism products. Prepare table of the above-mentioned measured values and their corresponding years, and connect the obtained point data into line to obtain the picture and database of the residual products of aspergillus and other microbes corresponding to the year of the ancient jade. Under the same conditions, measure the corresponding parameters of aspergillus and other microbes on the ancient jade to be identified, compare the obtained corresponding pictures, and then it is possible to know the production year corresponding to the ancient jade, thus the ancient jade identification is completed.

Method of this invention can be used for the jade ware, porcelain ware, bronze ware, calligraphy and painting, silk woven articles, as well as for criminal reconnaissance to solve the puzzle of the above-mentioned time and year. Also, it can be used in cosmetology (such as clearing up yellow spot and fleck) and for prevention and cure of human disease.

What is claimed is:

1. A method for identification of age of an ancient ware comprising the following steps:
A. establishing database corresponding to microbe parameters in the ware;
B. taking sample from the ware;
C. measuring values of microbe parameters in the sample from the ware;
D. comparing the values of microbe parameters obtained from the measuring with the established database to obtain age of the ware.

2. The method of claim 1, wherein the microbe is aspergillus.

3. The method of claim 1, wherein the microbe parameters are physical and/or chemical parameters of debris of the microbe.

4. The method of claim 1, wherein the microbe parameters are color change parameters of debris of the microbe.

5. The method of claim 1, wherein the microbe parameters are corrosion parameters of debris of the microbe.

6. The method of claim 1, wherein the values of microbe parameters are measured by using electronic microscopic scanner.

7. The method of claim 1, wherein the values of microbe parameters are measured by using artificial intelligence or human intelligence technology for judgment and quantification.
